SEQUENCE LISTING (7 sequences)

SEQ ID NO: 1 - DNA, 1779 bp, Gossypium hirsutum
  source 1..1779: mol_type = other DNA
atggctgcttttgttggggcagtgaagctctttatctctttggttctttggctcaacactctctcatctattcaaggtgcgagggatttctatgaatttcggtacccgttcatcaaaagggcgagttcattttcatcatcgtcatcgttctcatcatcgagtaacgatgaagttacgtacgactacataatcgtgggaggtggcacagccgggtgtcccttagcagccacactgtctcaaaacttcagtgtattgctgttggagagaggtggtgttcccttctcaaatgccaatgtctccttcctcagaaacttccacatcgccttggctgacacttccccaacctctgcttcccaacctttcgtttccaccgatggtgttattaatgctagggctagggttttgggtggtggcacttgcattaatgctggcttttataccagagctaactcagatttcatacggagagtgggatgggatgctaggctagtgaacgagtcctatccatgggtggagaaacagattgttcaccagcctaaactagcaccatggcaagatgccttcaaggacagtcttttagatgttggggtctcaccttataatggattcacctacgaccatatctatggaactaaagttggaggcaccattttcgaccgatttggccgtcgtcatacggcggctgagcttcttgcttctgctaacccgaaaatgcttactgttttagtgtatgccactgtccaaaaagtcttattcgataaatcagcagggaagaggccaaaggcaatgggagtgatgttcaaagatgaaaatgggaaccaacatcaggcatttttgactaataatcgaaggagtgaagtgattttatcatgtggtgcaattgggactcctcaattgctaatgcttagtggcattggacccaaagctgaactccagaggttgaacatttccatggtgttgcataatgagtttgttggaaagggcatggctgataacccgatgaactcggttttcgtcccgatgacacgttctgtggaacaatctcttatacaaactgttggcattaccaagatgggtgtttacatcgaagctagcagtggattcggacagtcccaggacagcattcattgtcatcatggattgttgtctgctgagatagggcaactctcaacagttccaccaaagcaaagaacacgacaagccatcgaagaattcatcaaaaggaaacgagaccttccacgtgaggctttcaagggaggtttcattttagagaagattgcaatgccactttccactggccacctcaacctaatcaacacaaacatcgacaacaacccttcggttaccttcaactacttcggccacccccgcgacctacgcagatgcgtcaatgggattcggatggctgccaaggttatacaatcggaccgattcacgaacttcaccaagtgtgacaaaccgaccgtagagaggcttcttaacatgagtgtcaatgctaacattaaccttatacccaagcatactaatgacaccaagtccctagaacagttctgcaaagacactgtaatcacaatctggcattaccatggagggtgccatgttgggaaggtggtggaccctaaccacaaagttcttggaaccagaagactccggatcgtggatggctccacatttagtgaatcccctggaactaaccctcaaggcactgtcttgatgatgggcaggtatatgggagtcaagattttgagacgaagactaggcaaagctgctggtgtgtaa

SEQ ID NO: 2 - DNA, 3203 bp, Gossypium hirsutum
  source 1..3203: mol_type = genomic DNA
atggctgcttttgttggggcagtgaagctctttatctctttggttctttggctcaacactctctcatctattcaaggttttcccttcttctctcatctctttttcttttcccatctttctccactgtttctgcatatatatatatatatatatatttatatcatctccttcttgttttgttttttgggtatatatagtttttcttaatctatataagtttatttacatatgttccttcttcaatataatatgcattttgtttctggaaaatttgggaagaaattaatgtcaactgtgatcccctattttccttttgttttatttttctacaatttacttccttttaattaaatggggtagttagatatcttttcatttcaacctttttcttttttcttttttttttgaatcttttcatttgaaatgaattatcaattatgttcataatgcctcattttttctagtactaatagtttcaattaaactactaatagttctcactaggtgttggagaagtaataacagtttttctgacggaataatttctgtcagaagatgtatgatgtatttccgacgatacttcctcttttgcgacggaaagcacccaattttttttgtgacgaaagtatagcgcgtcataaaatatgtatatataacaatatgaaaattttgctgcaaaattaattcaggtgcgagggatttctatgaatttcggtacccgttcatcaaaagggcgagttcattttcatcatcgtcatcgttctcatcatcgagtaacgatgaagttacgtacgactacataatcgtgggaggtggcacagccgggtgtcccttagcagccacactgtctcaaaacttcagtgtattgctgttggagagaggtggtgttcccttctcaaatgccaatgtctccttcctcagaaacttccacatcgccttggctgacacttccccaacctctgcttcccaacctttcgtttccaccgatggtgttattaatgctagggctagggttttgggtggtggcacttgcattaatgctggcttttataccagagctaactcagagtatgttagtttctcctcttcccttttttttttttttacatttgaacttaatacggttgcattaaggtcctaatgatctctgaaagtgactgtaaattatcctgatgttgaagaaaaaaagttcaaatataagagaatttggtccaacaatagttgacttttctatatacatgtgaaacctgaaaatcattgagtattgtccacatttattaagtatttaggtcccccatatcgccaaaattcttaaattagttggtgatcccatttaaatttctttttaattaaaatcaaggatgtagatgtttcggttatgaagaacaattgattaggtttttgttatgaattgtaaaactggaaaaataaaaagaacccattaatgattccatcttcctttctatgaagtcattttttcttccttaaatattcttatttttctttaaatttttctcccttttattgtgtcacctattaaatgtttggatgtgatattgagtggtccttctgccttaatttgcttgattcagagatatgattaggtaaggtctagtacagattccaaaatagaaagcaatgacatttatttgtacttcattattagcctatacatttaatttggtacaccctactaacattatgatttactaggcacaagcaccaccaagtggattttttttttttatgaaatttatggctcaaatgttgcagtttcatacggagagtgggatgggatgctaggctagtgaacgagtcctatccatgggtggagaaacagattgttcaccagcctaaactagcaccatggcaagatgccttcaaggacagtcttttagatgttggggtctcaccttataatggattcacctacgaccatatctatggaactaaagttggaggcaccattttcgaccgatttggccgtcgtcatacggcggctgagcttcttgcttctgctaacccgaaaatgcttactgttttagtgtatgccactgtccaaaaagtcttattcgataaatcagcagggaagaggccaaaggcaatgggagtgatgttcaaagatgaaaatgggaaccaacatcaggcatttttgactaataatcgaaggagtgaagtgattttatcatgtggtgcaattgggactcctcaattgctaatgcttagtggcattggacccaaagctgaactccagaggttgaacatttccatggtgttgcataatgagtttgttggaaagggcatggctgataacccgatgaactcggttttcgtcccgatgacacgttctgtggaacaatctcttatacaaactgttggcattaccaagatgggtgtttacatcgaagctagcagtggattcggacagtcccaggacagcattcattgtcatcatggattgttgtctgctgagatagggcaactctcaacagttccaccaaagcaaagaacacgacaagccatcgaagaattcatcaaaaggaaacgagaccttccacgtgaggctttcaagggaggtttcattttagagaagattgcaatgccactttccactggccacctcaacctaatcaacacaaacatcgacaacaacccttcggttaccttcaactacttcggccacccccgcgacctacgcagatgcgtcaatgggattcggatggctgccaaggttatacaatcggaccgattcacgaacttcaccaagtgtgacaaaccgaccgtagagaggcttcttaacatgagtgtcaatgctaacattaaccttatacccaagcatactaatgacaccaagtccctagaacagttctgcaaagacactgtaatcacaatctggcattaccatggagggtgccatgttgggaaggtggtggaccctaaccacaaagttcttggaaccagaagactccggatcgtggatggctccacatttagtgaatcccctggaactaaccctcaaggcactgtcttgatgatgggcaggtaagcattaaaggcacatttgaatagtaacacaaagcattaatcaactatgtacaaaagtggtagggcaagggcaaacactcattttctttttggatttatgcaggtatatgggagtcaagattttgagacgaagactaggcaaagctgctggtgtgtaa

SEQ ID NO: 3 - DNA, 20 bp, synthetic construct
  source 1..20: mol_type = other DNA
atggctgcttttgttggggc

SEQ ID NO: 4 - DNA, 22 bp, synthetic construct
  source 1..22: mol_type = other DNA
ttacacaccagcagctttgcct

SEQ ID NO: 5 - DNA, 20 bp, synthetic construct
  source 1..20: mol_type = other DNA
agggaacaccacctctctcc

SEQ ID NO: 6 - DNA, 22 bp, unidentified
  source 1..22: mol_type = other DNA
catcatcgtcatcgttctcatc

SEQ ID NO: 7 - DNA, 22 bp, unidentified
  source 1..22: mol_type = other DNA
catcaccgtcatcgttctcatc

(19) United States
(12) Patent Application Publication
(10) Pub. No.: US 2026/0009091 A1
(43) Pub. Date: Jan. 8, 2026

(54) APPLICATION OF GLUCOSE-METHANOL-CHOLINE (GMC) OXIDOREDUCTASE GENE ASSOCIATED WITH OIL AND PROTEIN QUALITY OF GOSSYPIUM HIRSUTUM

(71) Applicant: ZHEJIANG UNIVERSITY, Hangzhou (CN)

(72) Inventors: Lei FANG, Hangzhou (CN); Qian DENG, Hangzhou (CN); Ting ZHAO, Hangzhou (CN); Yan HU, Hangzhou (CN); Tianzhen ZHANG, Hangzhou (CN)

(73) Assignee: ZHEJIANG UNIVERSITY, Hangzhou (CN)

(21) Appl. No.: 19/330,890

(22) Filed: Sep. 17, 2025

Related U.S. Application Data
(63) Continuation of application No. PCT/CN2024/093240, filed on May 15, 2024.

(30) Foreign Application Priority Data
May 15, 2023 (CN) 202310542084.0

(51) Int. Cl. C12Q 1/6895

(52) U.S. Cl. CPC C12Q 1/6895; C12Q 2600/13; C12Q 2600/156

(57) ABSTRACT

An application of a GMC oxidoreductase gene associated with oil and protein quality of Gossypium hirsutum and the cDNA sequence SEQ ID NO: 1 and the genome sequence SEQ ID NO: 2 of the gene in tetraploid Gossypium hirsutum are provided. The gene is obtained by re-sequencing Gossypium hirsutum varieties and GWAS, and is significantly associated with the oil and protein content of cottonseed kernel in cotton. The SNP genotype of the gene is used to distinguish high-quality and low-quality haplotypes. The variety population is classified according to these two haplotypes, and statistical analysis is performed in combination with quality traits, which also proves that there is a significant correlation between GhGMC and the oil and protein content of cottonseed kernel in cotton.

### CROSS REFERENCE TO THE RELATED APPLICATIONS

This application is a continuation application of International Application No. PCT/CN2024/093240, filed on May 15, 2024, which is based upon and claims priority to Chinese Patent Application No. 202310542084.0, filed on May 15, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBHZQS-QQW005-PKGG_SequenceListing.xml, created on Jun. 24, 2025, and is 12,098 bytes in size.

### TECHNICAL FIELD

The present invention belongs to the field of biotechnology application and relates to an oxidoreductase gene associated with oil and protein content of cotton and application thereof.

### BACKGROUND

Cotton is an important economic crop, and cotton production has an important impact on the development of Chinese agriculture and even the national economy. Cottonseed kernel is a huge oil and protein resource; its production and processing play a significant role in alleviating Chinese edible oil pressure, and its economic benefits are gradually gaining attention. Studies have shown that there is a significant negative correlation between cottonseed kernel protein content and oil content. It is challenging to breed varieties with both high oil content and high protein content in the cottonseed kernel, but it is feasible to breed new cotton varieties with either high oil content or high protein content. Therefore, it is necessary to breed cotton varieties with high oil or high protein by exploring and utilizing excellent genetic variations related to oil and protein quality of cotton.

Due to the complexity of the cotton genome, the previous research on cotton quality traits mainly focused on quantitative trait locus (QTL) and other aspects. Genome-wide association study (GWAS) refers to using whole-genome re-sequencing technology to sequence each individual in a population with rich genetic diversity, obtaining millions of molecular markers (such as single nucleotide polymorphism (SNP) markers, copy number variation (CNV) markers), and conducting joint analysis with the target trait phenotypic data. Based on certain statistical methods, GWAS is performed to determine the association between mutation sites and species traits and to quickly obtain chromosome regions or gene loci that affect the phenotypic variation of target traits. With the rapid development of sequencing technology and computing methods, GWAS has now become a powerful tool for detecting natural variation in complex traits of crops (Rafalski et al., 2010). GWAS can mine genes related to a variety of agronomic traits on a large scale without the need to assume candidate genes in advance. GWAS has strong detection capabilities and high accuracy and has become a hot spot in molecular breeding research. At present, GWAS research has been successfully carried out in many crops, including rice, Chinese mustard, soybeans, and cotton, and has achieved fruitful results. Zhou et al. (2021) identified the resistance of 1520 rice varieties and 17 wild rice materials to three biotypes of brown planthopper and identified 3502 associated SNPs and 59 associated loci through GWAS analysis. The results showed that in order to resist the more harmful brown planthopper, the rice population mobilized more loci and variants. This study reveals the genetic basis of the interaction between and evolution of rice and brown planthopper. Kang et al. (2021) conducted a GWAS on flowering time and seed weight and found 12 common candidate genes that control flowering period. Haplotype analysis of two of the candidate genes, VIN3 and SRR1, found that they co-evolved during the spread and domestication of Chinese mustard, revealing the adaptation of Chinese mustard to the environment in the flowering period. Zhang et al. (2021) first re-sequenced 496 soybean core germplasms from all over the world, constructed a natural variation population, and found extensive variations among varieties in this population. Further, through GWAS, combined with genetic analysis and molecular biological methods, an R gene (named GmNNL1) with a Toll/interleukin-1 receptor-nucleotide binding site-leucine-rich repeat (TIR-NBS-LRR) domain was located and proved to have an important effect on the number of root nodules. Subsequent studies on this R gene revealed the genetic and molecular mechanisms of host-parasite compatibility during the interaction between soybean and rhizobia. In 2017, Fang et al. performed whole-genome re-sequencing on 318 Gossypium hirsutum materials, selected 258 varieties for GWAS analysis, and identified a total of 119 association loci, including 71 yield-related association loci, 45 fiber quality-related loci, and 3 loci related to resistance to Verticillium wilt. At the same time, two gene loci related to the ethylene pathway were found to be significantly correlated with yield (Fang L, et al., 2017b). Ma Zhiying's team re-sequenced 419 core germplasms of Gossypium hirsutum materials and conducted GWAS analysis on 13 fiber-related traits, identifying new genes related to flowering time, fiber length, and fiber strength, and found that the selection of fiber-related traits during cotton domestication and breeding increased the frequency of excellent alleles (Ma et al., 2018). The year before last, the team completed deep re-sequencing of 1,081 Gossypium hirsutum germplasm resources from all over the world and obtained 304,630 structural variants. Combined with the phenotypic data of fiber length, specific strength, micronaire, boll weight, lint percentage, and seed index obtained from large-scale environmental assessments and the data on resistance to Verticillium wilt of 401 resources, a GWAS was conducted, and 346 important structural variants were found to be significantly associated with quality, 97 with yield, and 3 with resistance to Verticillium wilt (Ma et al., 2021). Zhang Xianlong's team systematically compared the genomic differences between wild cotton species and domesticated cotton species for the first time and used GWAS to identify key loci that control cotton fiber quality-related traits. And the expression regulation mechanism of these genetic loci was explored combined with the transcriptome data of fiber development (Wang et al., 2017). The above research results fully demonstrate that GWAS has a high positioning accuracy, even reaching the level of a single gene. Using the obtained functional markers related to the target trait to screen the target trait will greatly accelerate the breeding process and efficiency and promote the rapid development of precision crop breeding.

Members of the glucose-methanol-choline (GMC) oxidoreductase family include oxidoreductases, dehydrogenases, lyases, and oxidases, which generally catalyze the oxidation of inactive alcohols to produce aldehydes or ketones (Dreveny et al., 2001). Arabidopsis ACE/HTH (AT1g72970) is a single-domain protein with a GMC oxidoreductase (pfam00732) domain and is thought to encode an α-alcohol dehydrogenase that catalyzes the biosynthesis of long-chain α-, Ω-dicarboxylic acids. This gene is specifically expressed in Arabidopsis epidermal cells, and its loss of function leads to disruption of the cuticle membrane structure (Krolikowski et al. 2003;Kurdyukov et al. 2006). Oryza sativa No Pollen 1 (OsNP1) and HOTHEAD-like 1 (HTH1) in rice and Irregular Pollen Exine 1 (IPE1) in maize all belong to the GMC oxidoreductase superfamily genes. These genes are believed to be involved in the oxidation of C16/C18Ω-hydroxy fatty acids, and single gene homozygous mutations of the gene can cause male sterility phenotypes without affecting other traits (Chang et al., 2016; Chen et al., 2017; Xu et al., 2017).

### SUMMARY

The purpose of the present invention is to mine a GMC oxidoreductase gene, which is simultaneously associated with the oil and protein content traits of the cottonseed kernel in Gossypium hirsutum through re-sequencing of the Gossypium hirsutum variety population and the GWAS. The results of the GWAS show that the gene is closely associated with the two important quality traits of oil and protein content of cottonseed kernel.

The purpose of the present invention can be achieved by the following technical solution:

Acquisition of the sequence of the GMC oxidoreductase gene GhGMC. The cDNA sequence in the tetraploid Gossypium hirsutum TM-1 is shown in SEQ ID NO: 1, and the genome sequence is shown in SEQ ID NO: 2. There are 3 introns in the gene, and the genome sequence is forward transcribed.

Application of the GMS oxidoreductase gene GhGMC in identifying high-oil and high-protein Gossypium hirsutum varieties. The population is divided into two haplotypes by the SNP genotype in the variety population. The SNP (D10Gh: 5700620) base of the cotton material with high oil and low protein is T; the SNP of the cotton material with low oil and high protein is C. Therefore, the cotton varieties with high oil and low protein and the cotton varieties with low oil and high protein can be quickly identified through the SNP locus. The SNP locus is located at 752-bp position of the GMC oxidoreductase gene. Therefore, a base at the 752-bp position of the GMC oxidoreductase gene in the Gossypium hirsutum plant is detected, wherein a cotton with a base T at the 752-bp position is a high-protein and low-oil cotton plant, and a cotton with a base C at the 752-bp position is a low-protein and high-oil cotton plant.

Further, primers for detecting the base at 752-bp position of the GMC oxidoreductase gene include the forward primer as shown in SEQ ID NO: 3 and the reverse primer as shown in SEQ ID NO: 5.

Application of the GMC oxidoreductase gene GhGMC in improving oil and protein content traits in cotton.

Application of the GMC oxidoreductase gene GhGMC in breeding new varieties of high-oil and high-protein cotton by genetic engineering.

The advantages of the present invention are as follows:
(1) The genome of allotetraploid cultivated cotton is relatively complex, and the research on Gossypium hirsutum resource mining and breeding is not in-depth enough. Based on the high-quality Gossypium hirsutum genome sequence, the present invention uses population genome re-sequencing and GWAS technologies to identify genes closely associated with oil and protein content traits of cottonseed kernel in Gossypium hirsutum. This technology has been widely used in research of different crops such as rice.
(2) The gene is a GMC oxidoreductase gene, which is a member of the GMC family. The GMC oxidoreductase gene GhGMC of the present invention is significantly associated with the two quality traits of oil and protein content of cottonseed kernel in the GWAS.
(3) The GhGMC cDNA and genomic sequence provided by the present invention were obtained by polymerase chain reaction (PCR) technology, which has the advantages of high specificity, high sensitivity, rapidity, and simplicity.
(4) GhGMC is highly expressed at different stages of cotton ovule development, and the expression level analysis is obtained by transcriptome sequencing. The results show that the gene is related to the formation of cottonseed kernel quality traits.
(5) The SNP genotypes of GhGMC in relatively high oil and protein variety populations and relatively low oil and protein variety populations are verified by PCR technology, which is easy to operate, highly sensitive, and accurate.
(6) According to the different SNP genotypes of GhGMC, the variety population can be divided into two major categories, and there are significant differences in the oil and protein content of cottonseed kernels between the two categories. The results further prove the correlation between the gene and the oil and protein content traits of cotton.

### BRIEF DESCRIPTION OF THE DRAWINGS

FIG. 1 shows GWAS results of oil and protein content traits of the cottonseed kernel in cotton.

Arrows indicate SNP loci associated with quality traits. The horizontal axis indicates the position on the chromosome (Mb), and the vertical axis indicates the significance of the SNP locus association, expressed as-logo (P value).

FIG. 2 shows expression levels of GhGMC in different tissues and developmental stages of cotton.

The horizontal axis represents different tissues, including root, stem, leaf, ovule, and fiber. Ovule tissue includes 3 days before flowering, 1 day before flowering, the day of flowering, and 1 to 35 days after flowering. Fiber tissue includes 5 to 25 days after flowering. The vertical axis indicates the relative expression level in fragments per kilobase of exon model per million mapped fragments (FPKM).

FIG. 3 shows sequence information of GhGMC and identification of different haplotypes.

A non-synonymous mutation SNP locus of the GhGMC sequence was detected in the variety population at 752-bp position of the genomic DNA sequence. The base of the genomic SNP locus mutates from T to C, and the corresponding position on the transcribed cDNA mutates from T to C, causing the amino acid to mutate from Ser to Pro. Where CATCATCGTCATCGTTCTCATC is shown in SEQ ID NO: 6, and CATCACCGTCATCGTTCTCATC is shown in SEQ ID NO: 7. The variety population is divided into different haplotypes, marked as GhGMCHOC IPC and GhGMCLOC HIPC. HPC represents high-oil, LPC represents low-oil, HOC represents high-protein, and LOC represents low-protein.

FIG. 4 shows a comparative analysis of oil and protein content between different haplotypes of GhGMC.

The box plot represents the distribution of oil content and protein content in the variety population. There are 48 varieties with the TT haplotype and 105 varieties with the CC haplotype. The white box plot (left) represents the distribution of quality traits of haplotype TT, and the gray box plot (right) represents the distribution of quality traits of haplotype CC. The horizontal line in the box represents the median of the trait distribution. ** indicates that there is a difference at the 0.01 level.

### DETAILED DESCRIPTION OF THE EMBODIMENTS

### (1) Mining of GMC Oxidoreductase Genes Associated With Oil and Protein Content of Cottonseed Kernel in Cotton

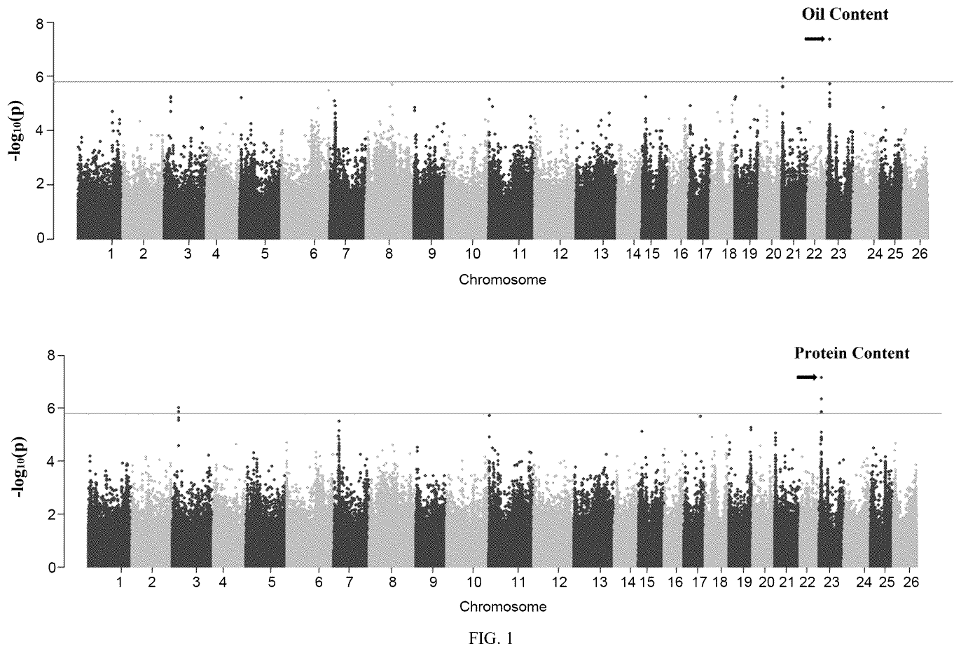

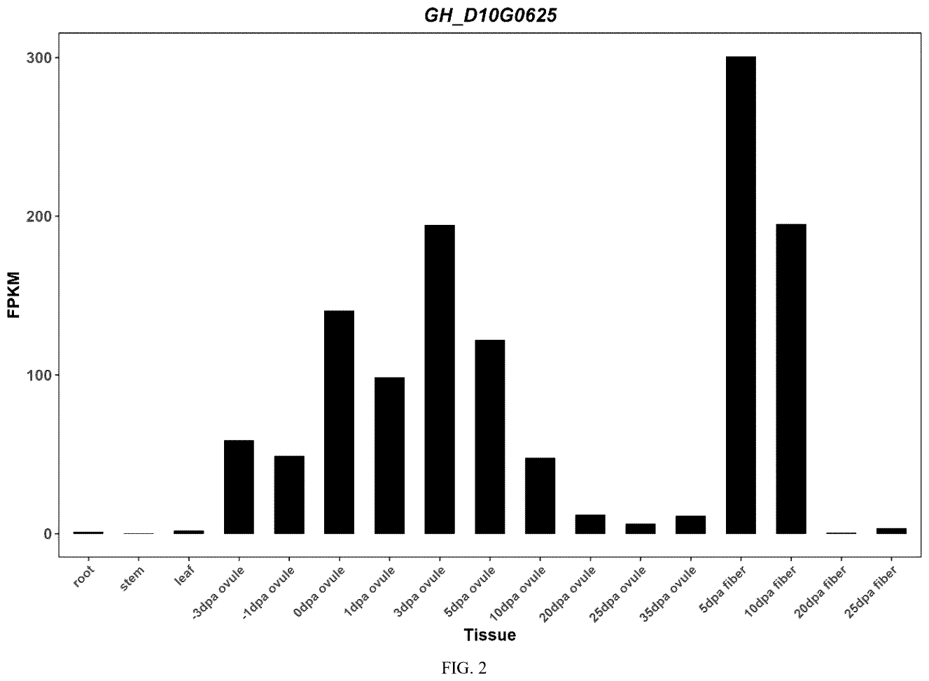

From 2007 to 2009, a detailed survey of oil and protein content traits of the cottonseed kernel was conducted in Anyang in Henan, Nanjing in Jiangsu, and Kuche in Xinjiang for 258 modern varieties or lines. At the same time, whole-genome re-sequencing was performed on the 258 cotton varieties to obtain 2.54 Tb of sequencing data with an average sequencing depth of 2.5×. These sequences were aligned to the genome sequence of TM-1 (V2.1), the genetic standard line of Gossypium hirsutum, and the whole genome SNP markers were identified using Samtools software. A total of 1,871,401 high-quality SNPs (minimum gene frequency >0.05) were mined for subsequent analysis. The EMMAx software was used to conduct the GWAS with the phenotype, and then the SNP association signal loci were screened according to P<1×10⁻⁶, and finally 71 cotton yield trait association loci were obtained. Among them, a trait-associated locus (D10Gh: 5687933) was identified on chromosome D10, which was significantly associated with both oil and protein content of the cottonseed kernel (FIG. 1). The candidate gene in the LD block region where this locus is located is the GMC oxidoreductase gene GhGMC (GH_D10G0625), and its cDNA sequence and genomic sequence are shown in SEQ ID NO: 1 and SEQ ID NO: 2.

### (2) Acquisition of the GMC Oxidoreductase Gene GhGMC

GhGMC (GH_D10G0625) was obtained from the genome sequence of Gossypium hirsutum. Primers of the gene were designed based on both ends of the cDNA for the full-length amplification. The primers are shown in Table 1. The PCR reaction procedure was as follows: pre-denaturation at 98° C. for 5 min; denaturation at 98° C. for 10 s, annealing at 58° C. for 5 s, extension at 68° C. for 25 s, 34 cycles; and finally extension at 68° C. for 5 min. The PCR amplification products were sequenced and compared with cDNA to determine the accuracy of the sequence. The GMC oxidoreductase gene GhGMC was obtained.

TABLE 1
PCR amplification primer sequences

| | Primer | Primer Sequence |
|---|---|---|
| Primer Name | Direction | Information |
| SEQ ID NO: 3 | Forward | ATGGCTGCTTTTGTTGGGGC |
| SEQ ID NO: 4 | Reverse | TTACACACCAGCAGCTTTGCCT |

### (3) Analysis of the Expression Level of GhGMC in Different Tissues and Developmental Stages of Cotton

This experiment used RNA samples from tissues such as root, stem, leaf, ovule, and fiber at different developmental stages for transcriptome sequencing, with an average sequencing read length of 25,530,023 for each sample. The reads obtained by sequencing were aligned with the Gossypium hirsutum genome TM-1 using TopHat 2.1.1 software, and the transcriptome data were quantified using Cufflinks 2.2.1 software. The expression level was expressed in FPKM (FIG. 2). The experimental results showed that the gene GhGMC was highly expressed at different stages of cotton ovule development, indicating that the gene was related to the formation of cottonseed kernel quality traits. The results show that GhGMC is indeed closely related to the key factors of the oil and protein content in cotton and has a significant effect on improving the oil and protein content of cottonseed kernel in cotton.

### (4) Application of GMC Oxidoreductase Gene in Identifying High-Oil and High-Protein Cotton

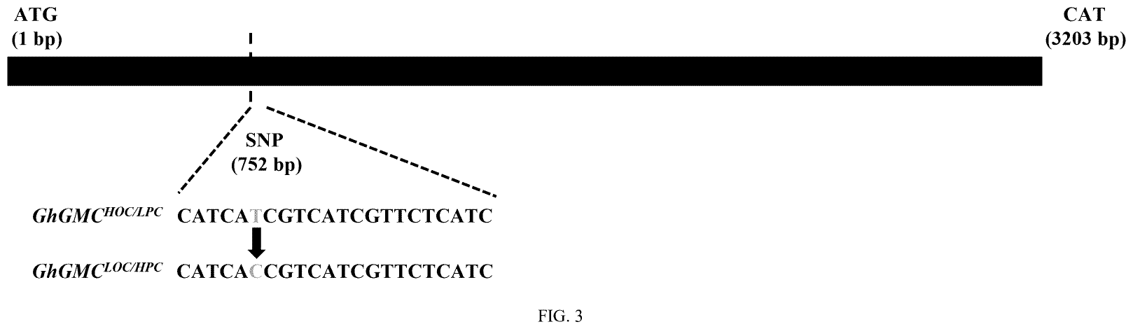

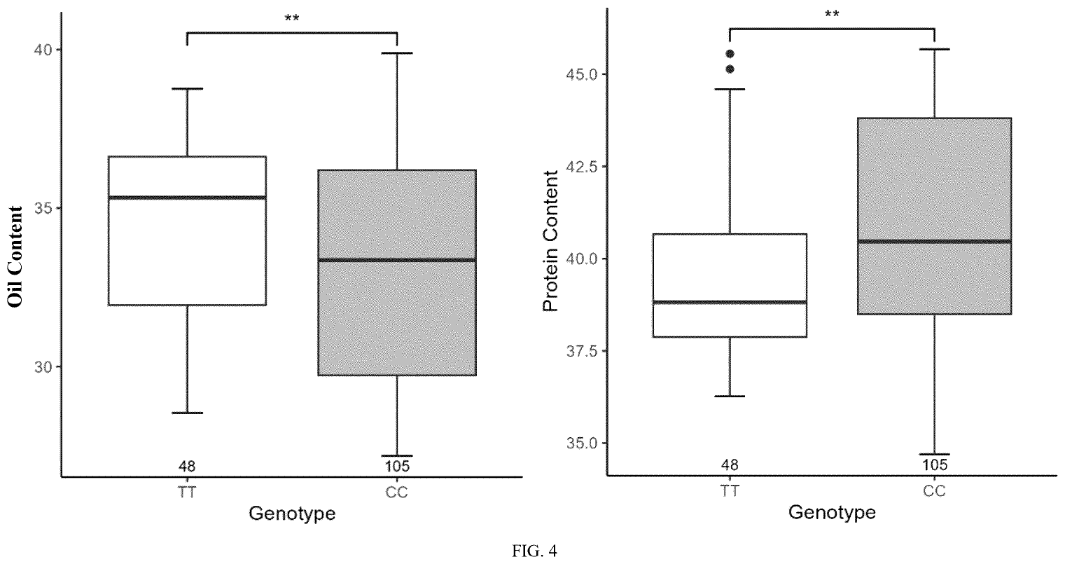

The GhGMC sequence has a non-synonymous mutation SNP locus in the population, as shown in FIG. 3. At 752-bp position of the genome sequence, the base mutates from T to C, and the corresponding position on the transcribed cDNA mutates from T to C, causing the amino acid to mutate from Ser to Pro. Based on the location of the SNP locus on chromosome D10 (D10Gh: 5700620), amplification primers (Table 2) were designed at both ends for PCR amplification and sequencing. The PCR reaction procedure was as follows: pre-denaturation at 98° C. for 5 min; denaturation at 98° C. for 10 s, annealing at 57° C. for 5 s, extension at 68° C. for 10 s, 34 cycles; and finally extension at 68° C. for 5 min.

TABLE 2
PCR amplification primer sequences

| | Primer | Primer Sequence |
|---|---|---|
| Primer Name | Direction | Information |
| SEQ ID NO: 3 | Forward | ATGGCTGCTTTTGTTGGGGC |
| SEQ ID NO: 5 | Reverse | AGGGAACACCACCTCTCTCC |

According to the base information and sequencing results of this SNP locus (D10: 5700620), the genotype of each variety population at this SNP locus was analyzed, and 48 haplotype TT materials and 105 haplotype CC materials were identified (Table 3). Combining the GWAS results and phenotypic survey data, the variety materials with TT haplotype were marked as GhGMC^{HOC/LPC}, and the variety materials with CC haplotype were marked as GhGMCLOC.HPC (FIG. 3). HPC represents high-protein, LPC represents low-protein; HOC represents high-oil, and LOC represents low-oil.

At the same time, the correlation between oil and protein content of cottonseed kernel between the two haplotypes was calculated using the t-test method (FIG. 4). The results show that compared with GhGMC^{LOC}(C), the oil content of haplotype GhGMC^{HOC}(T) increases by 4.73%, which is significantly positively correlated with the oil content trait (P=0.0082); the protein content of haplotype GhGMC^{HPC}(C) increases by 3.16%, which is significantly positively correlated with the protein content trait (P=0.0069).

The above results show that the gene GhGMC has important research value in improving the oil and protein content of cottonseed kernel in cotton and breeding new varieties of high-oil and high-protein cotton. On the one hand, molecular markers can be designed based on the two haplotypes of the gene GhGMC, which can effectively identify the oil and protein content traits of cottonseed kernel in cotton and have good application value in the research of breeding high oil and high protein cotton varieties. On the other hand, taking the breeding of new high-oil cotton varieties as an example, the gene containing the high-oil haplotype GhGMC(T) can be transferred into cotton varieties through genetic engineering to increase the oil content of cottonseed kernel in cotton, and the SNP loci in the low-oil haplotype GhGMC(C) can also be site-directed mutated to transform it into a high-oil haplotype to breed new high-oil cotton varieties.

TABLE 3
Distribution of high-oil and low-protein haplotype and low-oil
and high-protein haplotype in population variety materials

| Haplotype | Variety Name | | | |
|---|---|---|---|---|
| CC | M-8124-1159 | Australian L23/757 | Zhengzhou Long | Zhong 07 |
| | | | Staple Cotton | |
| | Xingtai 79-11 | J02-247 | Acala1517-2 | 601 Long Staple |
| | | | | Cotton |
| | Nashang District | Ji 91-22 | Xiang Cotton No. 2 | GK99-1 |
| | Large Flower | | | |
| | MM-2 | Qinli 514 | Zhong ARR40683- | Xushi Large Peach |
| | | | 4/RILNnXu0082F6 | |
| | Zhongzi 640 | Liao Cotton 17 | Zhong ARR40681 | Qifeng Large Bell |
| | Xinxiang 89S-210 | Acala (Large Bell)B | Large Bell Fuzi | Agricultural Resce |
| | | | Cotton | |
| | Suxu 138 | Shiyuan 638 | Xuzhou Semi-Semi | Ji 85-3 |
| | | | cotton | |
| | FB20 | Kuche T94-4 | GK20 | SGK Shixuan 321 |
| | TM-1 | L142-9 | Chad Cotton | Zhong Cotton |
| | | | | Institute No. 19 |
| | AcalaSJ-4 | USA 8123 | LAPAR45 | Shan 960329-2 Yuan 3 |
| | Zhong 117 | N73DeltapineNGF | Suyuan 04-129 | USA 28114-313 |
| | GP70 | Xuzhou 244 | Zhong 521 | Purple USA Cotton |
| | Ji Cotton25 | Zhongyuan 9115 | Zhongyuan 9114 | Soviet Cotton Series |
| | | | | 21 |
| | Jin 444 | 70-29-5 | Yahuang 9103 | Ji 91-31 |
| | Yangfen No. 31 | Australian Siv2 | GP83 | Zhongyuan 911 |
| | Zhong 89-1 | Jifeng 197 | E 408 | Soviet 8911 |
| | Kuche T94-1 | Su 7036 distant | Han 8944 | Qik |
| | Chaoyang No. 70 | Yancheng 1115 | Shan 3184 | Bazhou 7416 |
| | Zhong 1276 | Tu 188 | AcalaSJ-1 | Su TKH-1 |
| | Zhong AR40772 | Ji 91-18 | AcalaSJ-1-9 | Zhong G5 |
| | Liao Cotton 19 | Handan 568 | Yu Cotton2067 | Daze Cotton |
| | E Kang Cotton No. | Ji 668 | M11 | Qinyuan No. 4 |
| | 9 | | | |
| | Australian C | Handan 333 | Lu Cotton Yan 21 | Lu 458 |
| | Langhuang F10 | Su Q1 | Red Peach | Su Cotton 9108 |
| | Ji A-1-7 (Series 33) | 99633 | Zhongzi 4480 | r-3149 |
| | Ji Cotton No. 12 | E Jing 55173 | High Lint Cotton | Bao 6722 |
| | Tu 83-161 | | | |
| TT | Chad No. 3 | BPA68 | 353 Large Bell | MSCO-11 |
| | | | Series 3 | |
| | Su Cotton No. 2 | 0102X-10-1 | Liao 4835 | Yongji No. 2 |
| | Liao Cotton 18 | Xiaoxian Large Bell | Jiangsu Large Peach | Handan 109 |
| | Yun 93 Kang 393 | Zhong 12 | Zhong Cotton | Kang Huangwei 164 |
| | | | Institute 35 | |
| | Zhong 85271 | Yun 3060 | JinKang157 | Zhongzhi BD13 |
| | Ji 91-33 | Xuzhou 261 | Si 168 | Lu Cotton No. 11 |
| | Han 8959 | Sha 24-3 | Hu 749513 | Long Staple 67-12 |
| | Arcot436 | AC239 | GP138 | Large Bell Cotton |
| | | | | No. 69 |
| | Liao 61107 | GP95 | Kuche 93551 | Arcot-1(Original) |
| | DES926 | Liao 96-63-70 | MSCO-12 | upland |
| | USA F-19 | USA F-18 | GP93 | Jiyuan 12-13 |
| | USA D#1 | PAR-51 | Jin 90 Kang 282 | Jing 55263 |

Obviously, the above embodiments are merely examples for the purpose of clear explanation and are not intended to limit the implementation methods. For those skilled in the art, other different forms of changes or modifications can be made based on the above description. It is not necessary and impossible to list all the implementation methods here. The obvious changes or modifications derived therefrom are still within the scope of protection of the present invention.

Wat is claimed is:

1. A method for identifying a high-oil and high-protein cotton, comprising detecting a base at 752-bp position of a glucose-methanol-choline (GMC) oxidoreductase gene associated with an oil and protein quality of Gossypium hirsutum in a Gossypium hirsutum plant, wherein the nucleotide sequence of the GMC oxidoreductase gene is as shown in SEQ ID NO: 2; and a cotton with a base T at the 752-bp position is a high-protein and low-oil cotton plant, and a cotton with a base C at the 752-bp position is a low-protein and high-oil cotton plant.

2. The method according to claim 1, wherein primers for detecting the base at the 752-bp position of the GMC oxidoreductase gene comprise:
the forward primer as shown in SEQ ID NO: 3, and the reverse primer as shown in SEQ ID NO: 5.
